(19) United States
(12) Patent Application Publication
(10) Pub. No.: US 2010/0168433 A1
(43) Pub. Date: Jul. 1, 2010

(54) PROCESS FOR PREPARING BEPOTASTINE AND INTERMEDIATES USED THEREIN

(75) Inventors: Tae Hee Ha, Hwaseong-si (KR); Chang Hee Park, Yongin-si (KR); Won Jeoung Kim, Suwon-si (KR); Soohwa Cho, Seoul (KR); Han Kyong Kim, Yongin-si (KR); Kwee Hyun Suh, Suwon-si (KR)

Correspondence Address: SUGHRUE MION, PLLC, 2100 PENNSYLVANIA AVENUE, N.W., SUITE 800, WASHINGTON, DC, 20037, US

(73) Assignee: HANMI PHARM. CO., LTD., Hwaseong-si, Gyeonggi-do (KR)

(21) Appl. No.: 12/663,983

(22) PCT Filed: Jun. 5, 2008
(86) PCT No.: PCT/KR2008/003161

§ 371 Date: Dec. 10, 2009

(30) Foreign Application Priority Data
Jun. 11, 2007 (JP) 10-2007-0056740

(51) Int. Cl. C07D 401/12

(52) U.S. Cl. 546/194

(57) ABSTRACT

A process for stereospecific preparation of bepotastine of formula (I) and novel intermediates used therein having formulae (II) to (IV) are provided. The inventive process comprises subjecting (RS)-4-[(4-chlorophenyl)(2-pyridyl)methoxy]piperidine to a reaction with a 4-halobutanoic acid l-menthyl ester, halo being chloro, bromo or iodo, in an organic solvent in the presence of a base to produce (RS)-bepotastine l-menthyl ester of formula (II), conducting a reaction of the compound of formula (II) with N-benzyloxycarbonyl L-aspartic acid in an organic solvent to induce selective precipitation of bepotastine l-menthyl ester.N-benzyloxycarbonyl L-aspartate of formula (III), filtering the precipitates formed in step 2) to isolate the compound of formula (III), treating the compound of formula (III) with a base to liberate bepotastine l-menthyl ester of formula (IV), and hydrolyzing the compound of formula (IV) in the presence of a base. The inventive process can provide bepotastine having a high optical purity of not less than 99.5% in a high yield, and thus, is useful in the development of anti-histamines and anti-allergic agents.

### FIELD OF THE INVENTION

The present invention relates to a process for the stereospecific preparation of bepotastine and intermediates used therein.

### BACKGROUND OF THE INVENTION

Optically active bepotastine of formula (I), (+)-(S)-4-{4-[(4-chlorophenyl)(2-pyridyl)methoxy]piperidino}butyric acid, is a selective antihistamine as disclosed in JP 1998-237070.

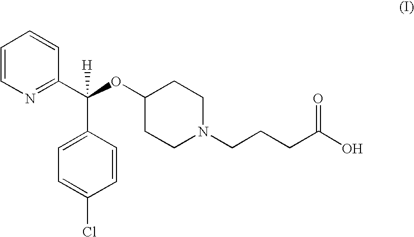

JP 1998-237070 and JP 2000-198784 disclose a preparation method of bepotastine as illustrated in Reaction Scheme 1, which comprises conducting optical resolution by treating racemic (RS)-4-[(4-chlorophenyl)(2-pyridyl)methoxy]piperidine (compound a) with optically active (2R,3R)-2-hydroxy-3-(4-methoxyphenyl)-3-(2-nitro-5-chlorophenylthio)propionic acid (compound b) to obtain the levorotatory isomer, (S)-(−)-4-[(4-chlorophenyl)(2-pyridyl)methoxy]piperidine (compound d) via compound c; and preparing bepotastine therefrom.

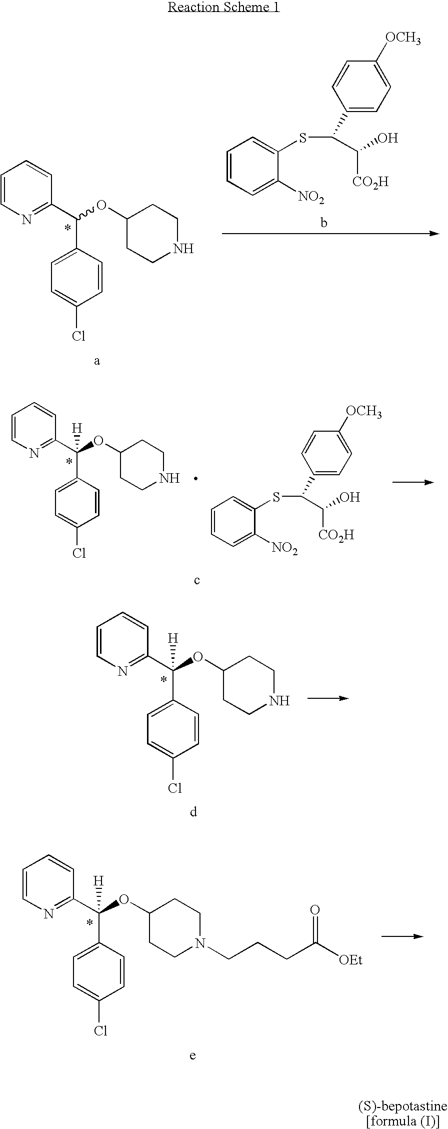

However, the above method is complicated and economically disadvantageous due to the fact that the preparation of compound b is required.

JP 2000-198784 describes a method for optically resolving racemic (RS)-4-[(4-chlorophenyl)(2-pyridyl)methoxy]piperidine (compound a of Reaction Scheme 1), by using N-acetyl-L-phenylalanine, N-acetyl-L-leucine, N-benzyloxycarbonyl-L-phenylalanine, N-benzyloxycarbonyl-L-valine, N-benzyloxycarbonyl-L-threonine or N-benzyloxycarbonyl-L-serine, among others, but the yield and the optical purity of the product obtained thereby are not satisfactory.

Meanwhile, JP 1998-237069 describes a method for recovering (RS)-4-[(4-chlorophenyl)(2-pyridyl)methoxy]piperidine (compound a) through the racemization of (R)-(+)-4-[(4-chlorophenyl)(2-pyridyl)methoxy]piperidine remaining in the filtrate after precipitating optically resolved (S)-(−)-4-[(4-chlorophenyl)(2-pyridyl)methoxy]piperidine (compound c). However, such a racemization requiring high temperature in butanol in the presence of base is not so efficient.

The present inventors have endeavored to develop an improved process for the stereospecific preparation of bepotastine and have found that bepotastine having a high optical purity can be prepared in a high yield by a method which uses novel intermediates such as (RS)-bepotastine l-menthyl ester, (S)-bepotastine l-menthyl ester.N-benzyloxycarbonyl L-aspartate and bepotastine l-menthyl ester.

### SUMMARY OF THE INVENTION

Accordingly, it is an object of the present invention to provide an efficient process for preparing high optical purity bepotastine in a highly stereospecific manner.

It is another object of the present invention to provide novel intermediates used in the above process.

### DETAILED DESCRIPTION OF THE INVENTION

In accordance with the present invention, there is provided a process for preparing bepotastine of formula (I) comprising the steps of:

1) subjecting (RS)-4-[(4-chlorophenyl)(2-pyridyl)methoxy]piperidine to a reaction with a 4-halobutanoic acid l-menthyl ester, halo being chloro, bromo or iodo, in an organic solvent in the presence of a base to produce (RS)-bepotastine l-menthyl ester of formula (II);

2) conducting a reaction of the compound of formula (II) with N-benzyloxycarbonyl L-aspartic acid in an organic solvent to induce selective precipitation of bepotastine l-menthyl ester N-benzyloxycarbonyl L-aspartate of formula (III);

3) filtering the precipitates formed in step 2) to isolate the compound of formula (III);

4) treating the compound of formula (III) with a base to liberate bepotastine l-menthyl ester of formula (IV); and

5) hydrolyzing the compound of formula (IV) in the presence of a base.

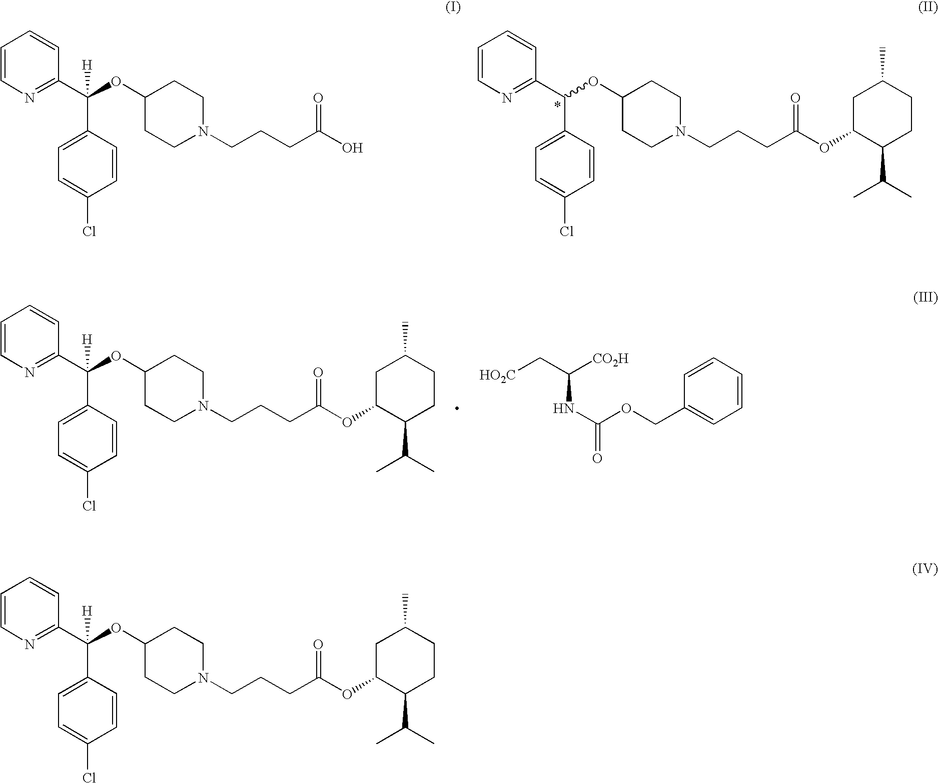

Each step of the inventive method is explained in detail below.

### Step 1) Preparation of racemic (RS)-bepotastine l-menthyl ester

In reaction step 1), (RS)-4-[(4-chlorophenyl)(2-pyridyl)methoxy]piperidine (see compound a of Reaction Scheme 1) prepared by the method described in U.S. Pat. No. 4,929,618 or another similar method is allowed to react with 4-halobutanoic acid l-menthyl ester (halo is chloro, bromo or iodo) in an organic solvent in the presence of a base to produce (RS)-bepotastine l-menthyl ester of formula (II).

The organic solvent used in the step 1) may be acetone, acetonitrile, ethyl acetate, tetrahydrofuran, benzene, toluene or N,N-dimethylformamide. The 4-halobutanoic acid l-menthyl ester may be used in an amount of 1 to 1.5 equivalents based on the (RS)-4-[(4-chlorophenyl)(2-pyridyl)methoxy]piperidine. The base may be triethylamine, diisopropyl ethylamine, potassium carbonate, sodium carbonate, potassium bicarbonate, or sodium bicarbonate, and it is used in an amount of 1 to 3 equivalents based on the 4-halobutanoic acid l-menthyl ester. The reaction is conducted at a temperature ranging 0° C. to the reflux temperature of the solvent.

### Step 2) Preparation of bepotastine l-menthyl ester.N-benzyloxycarbonyl L-aspartate (optical resolution)

In reaction step 2), (RS)-bepotastine l-menthyl ester of formula (II) obtained in step 1) is subjected to a reaction with N-benzyloxycarbonyl L-aspartic acid in an organic solvent, to induce the selective precipitation of bepotastine l-menthyl ester.N-benzyloxycarbonyl L-aspartate of formula (III).

N-benzyloxycarbonyl L-aspartic acid is used in an amount of 0.5 to 2.0 equivalents, more preferably, 1 to 1.2 equivalents based on the (RS)-bepotastine l-menthyl ester. The organic solvent may be acetonitrile, methyl ethyl ketone, methyl isobutyl ketone, methyl acetate, ethyl acetate, isopropyl acetate, diethyl ether or a mixture thereof, and preferably, methyl acetate or ethyl acetate. The amount of the organic solvent used is 3 to 30 ml per 1 g of (RS)-bepotastine l-menthyl ester. The reaction is carried out at a temperature of 10° C. to 60° C., and the reaction mixture is cooled to 5° C. to 20° C. The precipitated salt of formula (III) may be isolated therefrom by simple filtration.

### Steps 3) and 4) Preparation of bepotastine l-menthyl ester

Bepotastine l-menthyl ester.N-benzyloxycarbonyl L-aspartate is treated with a base to liberate bepotastine l-menthyl ester of formula (IV) only.

A weak base such as sodium bicarbonate and potassium bicarbonate may be used as the base in this step. The reaction may be conducted in a mixed solution of water and an organic solvent selected from ethyl acetate, dichloromethane, chloroform and diethyl ether at pH 7.5 to 9.0.

### Step 5) Preparation of bepotastine

In reaction step 5), bepotastine l-menthyl ester of formula (IV) is hydrolyzed in the presence of a base to give bepotastine.

Sodium hydroxide, potassium hydroxide and the like may be used as the base in an amount of 1 to 5 equivalents based on the bepotastine l-menthyl ester.

Such hydrolysis reaction may be carried out in a mixture of water and an organic solvent selected from methanol, ethanol, isopropanol, acetone, acetonitrile and tetrahydrofuran at a temperature of 10° C. to 60° C. Preferable water to the organic solvent mix ratio is 1:0.05 to 1:20.

Further, the present invention may further comprise the steps of recovering (R)-isomer-rich bepotastine l-menthyl ester from the filtrate obtained after filtering out the bepotastine l-menthyl ester.N-benzyloxycarbonyl L-aspartate of formula (III) precipitated in reaction step 2) and treating the recovered material with an acid to obtain fully racemized (RS)-bepotastine l-menthyl ester of formula (II).

To recover the (R) isomer-rich bepotastine l-menthyl ester from the filtrate, water is added to the filtrate, and the pH is adjusted to 7.5 to 9.0 by the addition of a weak base such as sodium bicarbonate and potassium bicarbonate. Then, the desired product is extracted therefrom using an organic solvent in accordance with a conventional method.

Then, the (R)-isomer-rich bepotastine l-menthyl ester is converted to fully racemized (RS)-bepotastine l-menthyl ester in an organic solvent selected from acetonitrile, methanol, ethanol and isopropanol, by treating with an organic acid such as acetic acid, propionic acid, and benzenesulfonic acid, at a temperature of from 60° C. to the reflux temperature of the solvent. The organic acid may be used in an amount of 3 to 15 equivalents based on the (R)-isomer-rich bepotastine l-menthyl ester. If acetic acid is used as the organic acid, the use of the organic solvent may be omitted. Preferably, the reaction time is within 12 hours.

Bepotastine prepared according to the inventive process may be converted to a pharmaceutically acceptable salt such as benzenesulfonate and calcium salt in accordance with any of the known methods (e.g., see Japanese Patent Laid-open Publication No. 1998-237070 and Korean Patent Application No. 2007-33756).

Further, the present invention provides novel intermediates used in the above preparation method, i.e., (RS)-bepotastine l-menthyl ester of formula (II), bepotastine l-menthyl ester.N-benzyloxycarbonyl L-aspartate of formula (III) and bepotastine l-menthyl ester of formula (IV).

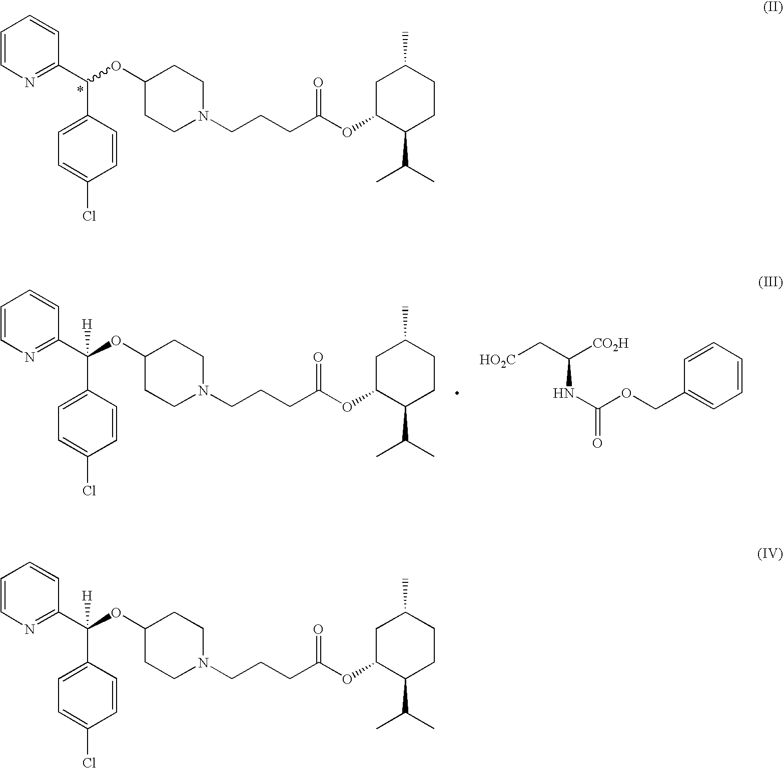

The present invention is explained in detail with reference to the Examples described below, which are given for the purpose of illustration only, and are not intended to limit the scope of the invention.

As discussed in the above, the inventive process of preparing bepotastine by using novel intermediates such as (RS)-bepotastine l-menthyl ester, bepotastine l-menthyl ester N-benzyloxycarbonyl L-aspartate and bepotastine l-menthyl ester can provide bepotastine having a high optical purity of not less than 99.5% in a high yield, and thus, is useful in the development of anti-histamines and anti-allergic agents.

### Reference Example

### Determination of the optical purity of a compound

In order to calculate the optical purity of each compound described in Examples, each isomer of the compound was isolated by conducting chromatography under the following conditions. The optical purity was calculated from the analysis results for each isomer based on Equation 1.
1) The conditions for analyzing the optical purity of bepotastine
   Detector: Ultraviolet absorption spectrophotometer (wave length for detection: 225 nm)
   Column: YMC Chiral 13-CDs (4.6×250 mm, 5 μm)
   Mobile phase: methanol/ammonium acetate buffer=45/55 (v/v′, %)
   Flow rate: 0.8 ml/min
2) The conditions for analyzing the optical purity of bepotastine l-menthyl ester
   Detector: Ultraviolet absorption spectrophotometer (wave length for detection: 230 nm)
   Column: ULTRON ES-OVM (4.6×150 mm, 5 μm)
   Mobile phase: acetonitrile/0.02M potassium dihydrogen phosphate=15/85 (v/v′, %)
   Flow rate: 1.0 ml/min

Optical purity (%)=Pₛ/(Pₛ+P_{R})×100 Equation 1

(Pₛ indicates the peak area of bepotastine or bepotastine l-menthyl ester, and P_{R} means the peak area of each corresponding (R)-isomer, both of which were obtained from chromatogram analysis.)

### Preparative Example 1

### Preparation of 4-bromobutanoic acid l-menthyl ester

14.6 g of l-menthol and 14.8 ml of pyridine were dissolved in 150 ml of dichloromethane, a solution obtained by dissolving 17.0 g of 4-bromobutyryl chloride in 20 ml of dichloromethane was slowly added dropwise thereto, and the resulting mixture was stirred at room temperature for 1 hour. The reaction mixture was washed with 100 ml of water, and the solvent was removed under a reduced pressure, to obtain 27 g (97%) of the title compound as an oil.

¹H-NMR (DMSO-d⁶, ppm): δ 4.7 (m, 1H), 3.5 (t, 2H), 2.5 (t, 2H), 2.2 (m, 2H), 2.0 (m, 1H), 1.9 (m, 1H), 1.7 (m, 2H), 1.5 (m, 1H), 1.3 (m, 1H), 1.1 (m, 3H), 0.9 (d, 6H), 0.7 (d, 3H).

IR (KBr, cm⁻¹): 2956, 2928, 2870, 1729, 1456, 1370, 1251, 1205, 1177, 1129, 984.

### Preparative Example 2

### Preparation of 4-chlorobutanoic acid l-menthyl ester

1.0 g of l-menthol and 1.0 ml of pyridine were dissolved in 5.0 ml of dichloromethane, a solution obtained by dissolving 0.7 ml of 4-chlorobutyryl chloride in 5.0 ml of dichloromethane was slowly added dropwise thereto, and the resulting mixture was stirred at room temperature for 1 hour. The reaction mixture was washed with 20 ml of water, and the solvent was removed under a reduced pressure, to obtain 1.6 g (99%) of the title compound as an oil.

¹H-NMR (DMSO-d⁶, ppm): δ 4.7 (m, 1H), 3.6 (t, 2H), 2.5 (t, 2H), 2.1 (m, 2H), 2.0 (m, 1H), 1.9 (m, 1H), 1.7 (m, 2H), 1.5 (m, 1H), 1.4 (m, 1H), 1.2 (m, 3H), 0.9 (d, 6H), 0.8 (d, 3H).

IR (KBr, cm⁻¹): 2956, 2929, 2869, 1729, 1456, 1386, 1371, 1308, 1204, 1177, 1010, 984, 964, 913.

### Example 1

### Preparation of racemic (RS)-bepotastine l-menthyl ester (the compound of formula (II))

24.0 g of (RS)-4-[(4-chlorophenyl)(2-pyridyl)methoxy]piperidine was dissolved in 240 ml of acetone, 27.0 g of 4-bromobutanoic acid l-menthyl ester obtained in Preparative Example 1 and 18.3 g of K₂CO₃ were sequentially added thereto, and the resulting mixture was refluxed for 7 hours. The reaction mixture was filtrated to remove insoluble solids, and the solvent was removed from the filtrate under a reduced pressure, to obtain 42.0 g (99%) of the title compound as an oil.

¹H-NMR (DMSO-d⁶, ppm): δ 8.5 (m, 1H), 7.7 (t, 1H), 7.5 (d, 1H), 7.4 (d, 2H), 7.3 (m, 2H), 7.2 (m, 1H), 5.6 (s, 1H), 4.7 (m, 1H), 3.5 (br. s, 1H), 2.7 (m, 2H), 2.3 (m, 4H), 2.1 (m, 1H), 2.0-1.6 (m, 11H), 1.5 (m, 1H), 1.4 (m, 1H), 1.2 (m, 3H), 0.9 (d, 6H), 0.7 (d, 3H).

IR (KBr, cm⁻¹): 2952, 2869, 2810, 1727, 1588, 1489, 1468, 1455, 1370, 1187, 1086, 984, 807, 768, 749.

### Example 2

### Preparation of racemic (RS)-bepotastine l-menthyl ester (the compound of formula (II))

1.0 g of 4-chlorobutanoic acid l-menthyl ester obtained in Preparative Example 2 and 1.25 g of sodium iodide were added to 10 ml of methyl isobutyl ketone, and the mixture was refluxed for 5 hours. To the resulting mixture, 1.0 g of (RS)-4-[(4-chlorophenyl)(2-pyridyl)methoxy]piperidine and 1.7 g of potassium carbonate were sequentially added, followed by refluxing for 1 hour. Then, 15 ml of water and 30 ml of ethyl acetate were added to the reaction mixture to carry out extraction. The organic layer was separated therefrom, and concentrated under a reduced pressure, to obtain 1.8 g (99%) of the title compound as an oil.

### Example 3

### Preparation of bepotastine l-menthyl ester.N-benzyloxycarbonyl L-aspartate (the compound of formula (III))

90 g of (RS)-bepotastine l-menthyl ester obtained in Example 1 was dissolved in 900 ml of ethyl acetate, 45.7 g of N-benzyloxycarbonyl L-aspartic acid was added thereto, and the resulting mixture was stirred at room temperature for 12 hours. The solid precipitates formed therein was filtered and dried to obtain 48.2 g (yield: 71%, optical purity: 89.7%) of the title compound as a white crystal.

45.0 g of the compound thus obtained was added to 450 ml of ethyl acetate, and the resulting mixture was fully dissolved by heating. The solution was slowly cooled to room temperature and stirred for 12 hours to induce solid precipitation. The solid was filtered and dried, to obtain 39.2 g (yield: 87%, optical purity: 96.7%) of the title compound as a white crystal.

36.0 g of the crude product thus obtained was recrystallized from ethyl acetate by repeating the above procedure to obtain 32.8 g (yield: 91%, optical purity: 99.5%) of the title compound as a white crystal.

Specific optical rotation: [α]_{D}²⁴−15.2 (c=1.0, MeOH)

Melting point: 108 110 (degradation)

¹H-NMR (DMSO-d⁶, ppm): δ 8.5 (d, 1H), 7.8 (t, 1H), 7.5 (d, 1H), 7.4-7.2 (m, 10H), 7.2 (m, 1H), 5.6 (s, 1H), 5.0 (s, 2H), 4.5 (m, 1H), 4.1 (m, 1H), 3.5 (br. s, 1H), 2.9 (br. m, 2H), 2.6-2.3 (m, 5H), 2.2 (t, 2H), 1.9-1.6 (m, 11H), 1.5 (m, 1H), 1.4 (m, 1H), 1.0 (m, 3H), 0.9 (d, 6H), 0.7 (d, 3H).

IR (KBr, cm⁻¹): 3412, 2956, 2928, 2870, 1725, 1592, 1491, 1455, 1435, 1389, 1227, 1191, 1068, 960, 772, 696, 673.

### Example 4

### Preparation of bepotastine l-menthyl ester.N-benzyloxycarbonyl L-aspartate (the compound of formula (III))

90.0 g of (RS)-bepotastine l-menthyl ester obtained in Example 1 was dissolved in 900 ml of ethyl acetate, 45.7 g of N-benzyloxycarbonyl L-aspartic acid was added thereto, and the resulting mixture was dissolved by heating at the boiling point of the solvent. The solution was slowly cooled to room temperature and stirred for 12 hours to induce solid precipitation. The solid was filtered and dried, to obtain 47.5 g (yield: 70%, optical purity: 95.2%) of the title compound as a white crystal.

### Example 5

### Preparation of bepotastine l-menthyl ester.N-benzyloxycarbonyl L-aspartate (the compound of formula (III))

90.0 g of (RS)-bepotastine l-menthyl ester obtained in Example 1 was dissolved in 900 ml of ethyl acetate, 45.7 g of N-benzyloxycarbonyl L-aspartic acid was added thereto and the resulting mixture was heated to the boiling point of the solvent to dissolve. The solution thus obtained was cooled slowly to room temperature, 0.5 g of bepotastine l-menthyl ester N-benzyloxycarbonyl L-aspartate obtained in Example 3 was added thereto, and stirred for 12 hours. The precipitate thus formed was filtered and dried, to obtain 49.5 g (yield: 73%, optical purity: 95.3%) of the title compound as a white crystal.

### Example 6

### Preparation of bepotastine l-menthyl ester (the compound of formula (IV))

30 g of bepotastine l-menthyl ester.N-benzyloxycarbonyl L-aspartate obtained in Example 3 was mixed with 300 ml of ethyl acetate and 200 ml of water and the pH of the resulting mixture was adjusted to 8.0 with saturated sodium bicarbonate to induce phase separation. Then, the organic layer was separated and the solvent was removed therefrom under a reduced pressure, to obtain 19.5 g (yield: 98%, optical purity: 99.5%) of the title compound as an oil.

¹H-NMR (DMSO-d⁶, ppm): δ 8.5 (m, 1H), 7.7 (t, 1H), 7.5 (d, 1H), 7.4 (d, 2H), 7.3 (m, 2H), 7.2 (m, 1H), 5.6 (s, 1H), 4.7 (m, 1H), 3.5 (br. s, 1H), 2.7 (m, 2H), 2.3 (m, 4H), 2.1 (m, 1H), 2.0-1.6 (m, 11H), 1.5 (m, 1H), 1.4 (m, 1H), 1.2 (m, 3H), 0.9 (d, 6H), 0.7 (d, 3H).

IR (KBr, cm⁻¹): 2953, 2869, 2811, 1728, 1588, 1489, 1469, 1456, 1434, 1370, 1253, 1188, 1108, 1086, 1015, 984, 807, 768, 749, 615.

### Example 7

### Preparation of Bepotastine

15.0 g of bepotastine l-menthyl ester obtained in Example 6 was dissolved in a mixture of 50 ml of ethanol and 50 ml of water, 3.4 g of sodium hydroxide was added thereto, and the resulting mixture was stirred at room temperature for 10 hours. After adding water, the resulting mixture was washed with ethyl ether, and 30 ml of 3N HCl was added to the aqueous solution, which was extracted with dichloromethane. The organic layer thus obtained was subjected to a reduced pressure to remove the solvent therefrom. As a result, 10.2 g (yield: 92%, optical purity: 99.5%) of the title compound was obtained in the form of a foam.

¹H-NMR (CDCl₃, ppm): δ 8.6 (d, 1H), 7.7 (t, 1H), 7.4 (d, 1H), 7.4-7.2 (m, 5H), 5.6 (s, 1H), 3.8 (br. s, 1H), 3.0 (t, 2H), 2.5 (m, 2H), 2.3 (m, 2H), 1.9 (m, 4H).

### Example 8

### Preparation of Bepotastine Benzenesulfonate

4.0 g of bepotastine obtained in Example 7 was dissolved in 40 ml of acetonitrile, and 1.5 g of benzenesulfonic acid monohydrate was added thereto. To the resulting mixture, 0.05 g of bepotastine benzenesulfonate obtained in accordance with the method described in U.S. Pat. No. 6,307,052 was added, followed by stirring at room temperature for 12 hours. The solid thus obtained was filtered to obtain 3.0 g (yield: 64%, optical purity: 99.5%) of the title compound as a pale white crystalline powder.

Melting point: 161˜163° C.

Water: 0.2% (Karl-Fischer water determination)

¹H-NMR (DMSO-d₆): δ 9.2 (bs, 1H), 8.5 (d, 1H), 7.8 (t, 1H), 7.6 (m, 3H), 7.4 (m, 4H), 7.3 (m, 4H), 5.7 (d, 1H), 3.7 (bs, 2H), 3.3 (bs, 3H), 3.1 (bs, 2H), 2.3 (t, 2H), 2.2 (m, 1H), 2.0 (m, 1H), 1.8 (m, 3H), 1.7 (m, 1H).

IR (KBr, cm⁻¹): 3422, 2996, 2909, 2735, 2690, 2628, 1719, 1592, 1572, 1488, 1470, 1436, 1411, 1320, 1274, 1221, 1160, 1123, 1066, 1031, 1014, 996, 849, 830, 771, 759, 727, 693, 612, 564.1.

### Example 9

### Preparation of Bepotastine Calcium Salt

4.0 g of bepotastine obtained in Example 7 was mixed with 2.2 ml of 5N aqueous sodium hydroxide solution and 20 ml of water, a solution obtained by dissolving 1.6 g of calcium chloride in 20 ml of water was slowly added dropwise thereto, and the resulting mixture was stirred at room temperature for 12 hours. The solid thus obtained was filtered to obtain 3.62 g (yield: 86%, optical purity: 99.5%) of the title compound as a white crystalline powder.

Water: 4.4% (Karl-Fischer water determination, a theoretical value of dihydrate 4.23%)

Melting point: 238˜240° C. (degradation)

¹H-NMR (DMSO-d⁶, ppm): δ 8.4 (d, 1H), 7.8 (t, 1H), 7.5 (d, 1H), 7.4 (m, 4H), 7.2 (t, 2H), 5.6 (s, 1H), 3.5 (m, 1H), 2.6 (m, 2H), 2.2 (t, 2H), 1.9 (m, 4H), 1.8 (m, 2H), 1.6 (m, 4H).

IR (KBr, cm⁻¹): 3338, 2945, 2825, 1589, 1562, 1490, 1471, 1432, 1412.9, 1308, 1116, 1092, 1061, 1014, 994, 808, 776, 750.

### Example 10

### Preparation of racemic (RS)-bepotastine l-menthyl ester (the compound of formula (II))

To the filtrate obtained after filtering the precipitate in Example 5, 600 ml of water was added, and a pH of the resulting mixture was adjusted to 8.0 with sodium bicarbonate. Then, the organic layer was separated therefrom, and concentrated to obtain 57 g of (R)-isomer-rich bepotastine l-menthyl ester ((R)-isomer:(S)-isomer=76:24) as an oil.

The (R)-isomer-rich bepotastine l-menthyl ester thus obtained was dissolved in 60 ml of acetic acid and refluxed for 3 hours, and then, 500 ml of water and 500 ml of ethyl acetate were added thereto. The organic layer was separated therefrom, washed with water and saturated sodium bicarbonate, and concentrated under a reduced pressure, to obtain 51 g (yield: 90%, (S)-isomer:(R)-isomer=49.9:50.1) of the title compound as an oil.

### Example 11

### Preparation of bepotastine l-menthyl ester.N-benzyloxycarbonyl L-aspartate (the compound of formula (III))

30.0 g of (RS)-bepotastine l-menthyl ester obtained in Example 10 was dissolved in 300 ml of ethyl acetate, 15.2 g of N-benzyloxycarbonyl L-aspartic acid was added thereto, and the resulting mixture was dissolved by heating at the boiling point of the solvent. The solution was slowly cooled to room temperature, and stirred for 12 hours to induce solid precipitation. The solid was filtered and dried, to obtain 15.4 g (yield: 68%, optical purity: 95.6%) of the title compound as a white crystal.

While the invention has been described with respect to the above specific embodiments, it should be recognized that various modifications and changes may be made to the invention by those skilled in the art which also fall within the scope of the invention as defined by the appended claims.

What is claimed is:

1. A process for preparing bepotastine of formula (I) comprising the steps of:
1) subjecting (RS)-4-[(4-chlorophenyl)(2-pyridyl)methoxy]piperidine to a reaction with a 4-halobutanoic acid l-menthyl ester, halo being chloro, bromo or iodo, in an organic solvent in the presence of a base to produce (RS)-bepotastine l-menthyl ester of formula (II);
2) conducting a reaction of the compound of formula (II) with N-benzyloxycarbonyl L-aspartic acid in an organic solvent to induce selective precipitation of bepotastine l-menthyl ester N-benzyloxycarbonyl L-aspartate of formula (III);
3) filtering the precipitates formed in step 2) to isolate the compound of formula (III); 4) treating the compound of formula (III) with a base to liberate bepotastine l-menthyl ester of formula (IV); and
5) hydrolyzing the compound of formula (IV) in the presence of a base.

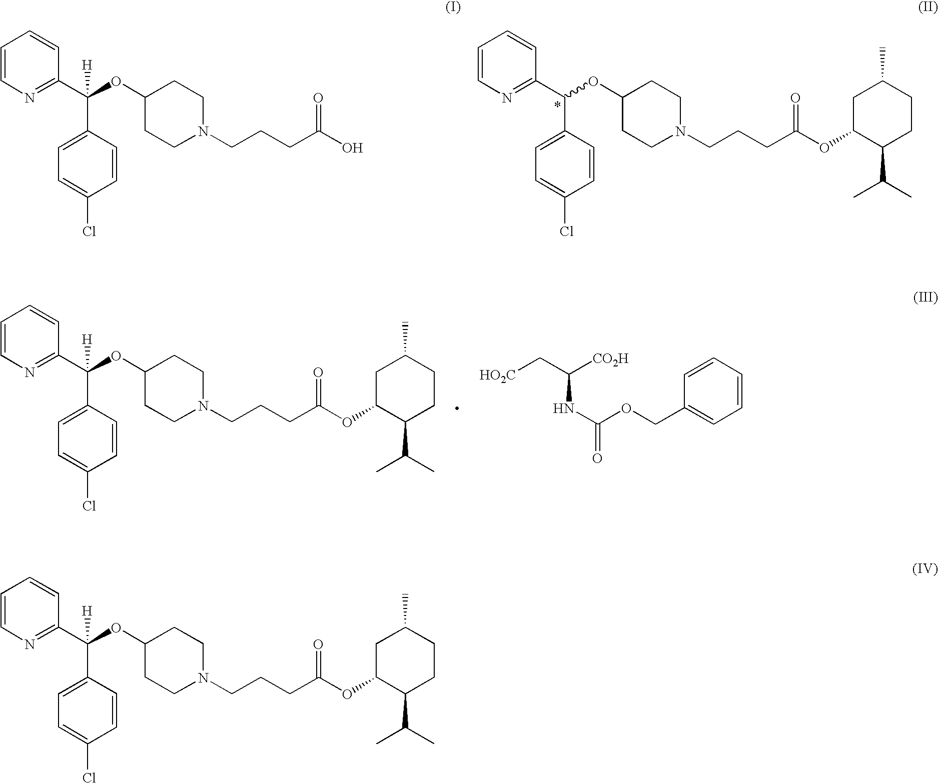

2. The process of claim 1, wherein the solvent used in step 1) is selected from the group consisting of acetone, acetonitrile, ethyl acetate, tetrahydrofuran, benzene, toluene, and N,N-dimethylformamide.

3. The process of claim 1, wherein the amount of the 4-halobutanoic acid l-menthyl ester used in step 1) is 1 to 1.5 equivalents based on the (RS)-4-[(4-chlorophenyl)(2-pyridyl)methoxy]piperidine.

4. The process of claim 1, wherein the base used in step 1) is selected from the group consisting of triethylamine, diisopropyl ethylamine, potassium carbonate, sodium carbonate, potassium bicarbonate, and sodium bicarbonate.

5. The process of claim 1, wherein the organic solvent used in step 2) is selected from the group consisting of acetonitrile, methyl ethyl ketone, methyl isobutyl ketone, methyl acetate, ethyl acetate, isopropyl acetate, and diethyl ether.

6. The process of claim 1, wherein the amount of the N-benzyloxycarbonyl L-aspartic acid used in step 2) is 0.5 to 2.0 equivalents based on the (RS)-bepotastine l-menthyl ester.

7. The process of claim 1, wherein the base used in step 4) is sodium bicarbonate or potassium bicarbonate.

8. The process of claim 1, wherein step 4) is conducted in a mixture of an organic solvent and water at a pH in the range of 7.5 to 9.0.

9. The process of claim 8, wherein the organic solvent is selected from the group consisting of ethyl acetate, dichloromethane, chloroform, and diethyl ether.

10. The process of claim 1, wherein the base used in step 5) is sodium hydroxide or potassium hydroxide.

11. The process of claim 1, wherein step 5) is conducted in a mixture of water and an organic solvent selected from the group consisting of methanol, ethanol, isopropanol, acetone, acetonitrile, and tetrahydrofuran.

12. The process of claim 1, further comprising the steps of recovering (R)-isomer-rich bepotastine l-menthyl ester by treating the filtrate generated in step 3) with a base, and treating the recovered material with an acid to obtain racemic (RS)-bepotastine l-menthyl ester of formula (II).

13. (RS)-bepotastine l-menthyl ester of formula (II) used as an intermediate in the process of claim 1.

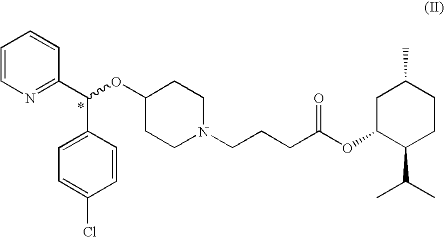

14. Bepotastine l-menthyl ester.N-benzyloxycarbonyl L-aspartate of formula (III) used as an intermediate in the process of claim 1.

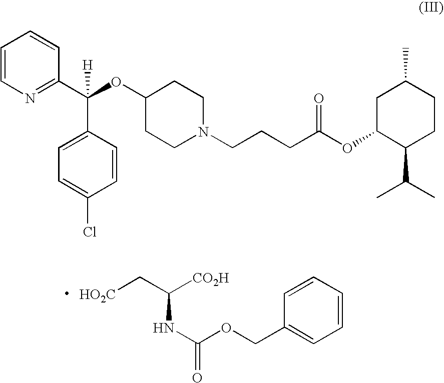

15. Bepotastine l-menthyl ester of formula (IV) used as an intermediate in the process of claim 1. [chemical structure]
